(19) United States
(12) Patent Application Publication
(10) Pub. No.: US 2025/0384972 A1
(43) Pub. Date: Dec. 18, 2025

(54) METHOD AND SYSTEM FOR IDENTIFYING OPTIMAL CLINICAL TRIAL DESIGN PARAMETERS USING MACHINE LEARNING TRAINED ON SIMULATION OUTCOMES

(71) Applicant: PHASEV TRIALS LTD, Tel Aviv (IL)

(72) Inventors: Raviv PRYLUK, Ramat Gan (IL); Elad BERKMAN, Givataim (IL); Amitai KAMBER, Givatayim (IL); Oshri MACHLUF, Ramat Gan (IL); Tzviel FROSTIG, Tel Aviv (IL); Roni COHEN, Tel Aviv (IL); Amitai LEVY, Raanana (IL)

(21) Appl. No.: 18/745,006

(22) Filed: Jun. 17, 2024

(51) Int. Cl. G16H 10/20; G16H 50/20

(52) U.S. Cl. CPC G16H 10/20; G16H 50/20; G16H 50/50

(57) ABSTRACT

Disclosed are method and system for identifying optimal clinical trial design parameters, the method including a first plurality of simulations for selected working points; training a machine learning model on the plurality of simulations and their respective simulated outcomes to obtain an ML model configured to output predicted simulation outcomes for non-simulated working points within the space of working points; and reiterating the process until an optimal set of working points and their associated parameters are obtained.

### TECHNICAL FIELD

The present disclosure relates generally to clinical trials, more specifically but not exclusively to methods and systems for identifying optimal clinical trial design parameters using machine learning trained on simulation outcomes.

### BACKGROUND

Clinical trials, particularly advanced and complex ones, face significant challenges due to the absence of straightforward analytical methods to calculate their operating characteristics.

Designers often need to conduct extensive simulation, which is not only expensive, but also slow, owing to the high volume of simulations needed for accuracy. Furthermore, complex trials typically have multiple degrees of freedom (e.g., number of interim analyses, timing of interims, and the like), making manual optimization nearly impossible without relying on heuristic methods, based on prior trials and subjective intuition.

Brute force simulation involves running a large number of simulations to calculate the operating characteristics of a trial under various configurations. This process is computationally intensive as each simulation might require substantial time and processing power, particularly when the trials are complex with many variables. This computational demand translates into higher costs, both in terms of the time invested by research teams and the financial cost associated with high-performance computing resources.

Each simulation provides estimates that are inherently uncertain. Therefore, to achieve reliable results, many iterations are needed. However, the more complex the trial design (e.g., adaptive trials with multiple interim analyses and multiple adaptations), the greater the number of simulations required to meet statistical requirements, such as type 1 error and the like. This can lead to inefficiencies, as the process becomes not only slower, but also less responsive to real-time data and adjustments.

As the number of variables increases (such as different dosage levels, timing of doses, or number of interim analyses), the dimensionality of the simulation space explodes. The complexity of design process can make it impractical to explore all potential combinations of trial parameters thoroughly, thereby limiting the scope of trial designs that can be feasibly evaluated.

Thus, there is a need to provide a method and system for calculating and estimating the operating parameters of clinical trials, in an efficient manner saving cost, time and computational resources, without compromising accuracy.

### SUMMARY

According to some embodiments, methods and systems for identifying optimal clinical trial design parameters using machine learning trained on simulation outcomes is presented herein.

According to some embodiments, the method disclosed herein employs a machine learning algorithm using results of a series of simulations to learn the relationship between a trial's design parameters and its operating characteristics. By integrating advanced computational techniques, the machine learning model efficiently maps out the multidimensional parameters space and supports the required optimization process.

According to some embodiments, the method of the present disclosure, presents a robust model that uses machine learning to estimate and predict the operating characteristics of clinical trials, while taking into consideration various design adjustments.

According to some embodiments, the method presented herein advantageously reduces the need for extensive simulations dramatically, achieving the required predictive accuracy with at least 25 times fewer simulations compared to brute force methods.

According to some embodiments, through rigorous training/testing methodologies, the machine learning algorithm model has been validated to ensure high accuracy and reliability in predicting trial outcomes.

According to some embodiments, the implementation of the machine learning model in clinical trial design advantageously enhances the speed and efficiency of trial setup significantly. By reducing the dependency on extensive simulations, trial designers can explore a broader array of design parameters more quickly and with greater precision. This not only saves time and resources but also potentially increases the efficacy and adaptability of clinical trials.

According to some embodiments, in one aspect, a method for identifying optimal clinical trial design parameters is presented herein. The method comprises:
a. receiving/inputting a plurality of optional clinical trial design parameters collectively defining a space of working points;
b. selecting a first subset of working points from within the first space of working points, wherein each working point is defined by a different set of clinical trial design parameters;
c. running a plurality of simulations for each of the selected working points to obtain their respective simulated outcome;
d. training an ML model on said plurality of simulations and their respective simulated outcomes to obtain an ML model configured to output predicted simulation outcomes for non-simulated working points within the space of working points;
e. applying the trained ML model on non-simulated working points within the space of working points, thereby mapping the space;
f. defining an improved space of working points based on the mapping and updating the trained ML model, based on simulated treatment outcomes of a first plurality of working points from the improved space of working points;
g. repeating steps b-g for the improved space of working points until obtaining an optimal space of working points;
h. identifying/outputting an optimal set of clinical trial design parameters, based on the predicted simulation outcomes of a second plurality of working points from the optimal space of working points.

According to some embodiments, the first plurality of simulations may include a small plurality of simulations, e.g. 2, 3, 4, 5, 6, 10, 20, 50, or 100 simulations (or any range therebetween). Each possibility is a separate embodiment. According to some embodiments, each additional iteration (step g) may include the same or a larger number of simulations (but less than 5000, and preferably less than 1000).

According to some embodiments, the method further comprises running 100000 simulations after identifying/outputting the optimal set of clinical trial design parameters, as may be required by regulations.

According to some embodiments, identifying/outputting an optimal set of clinical trial design parameters comprises optimizing sample size, cost of the clinical trial, duration of the clinical trial, estimated treatment efficacy of the trial, probability of success of the trial or any combination thereof.

According to some embodiments, the method further comprises outputting, for the optimal set of trial design parameters, one or more of: a probability of overall trial success, a probability of finding a best treatment as a function of the number of patients included in the trial, estimated distribution of cost and time of the trial overall, estimated distribution of cost and time until identification of failure, estimated distribution of cost and time until identification of success, distribution of estimated treatment effect, distribution of statistical measures.

According to some embodiments, at least a portion of the clinical and/or statistical input parameters comprise value ranges.

According to some embodiments, the value ranges are predetermined.

According to some embodiments, the method may further include determining/computing suitable ranges for the portion of clinical and/or statistical input parameters.

According to some embodiments, the selection of working points of step (b) is given and/or computed.

According to some embodiments, the number of simulations included in the plurality of simulations is predetermined.

According to some embodiments, the number of simulations included in the plurality of simulations is determined based on a number of simulations required to obtain an accuracy above a predetermined threshold.

According to some embodiments, the plurality of simulations comprises between 50 and 5000 simulations.

According to some embodiments, optional clinical trial design parameters comprise clinical and statistical input parameters.

According to some embodiments, the clinical parameters are selected from primary endpoint, delay, number of arms, futility threshold efficacy, efficacy threshold how good before deciding success assumed clinical efficacy, recruitment rate, primary endpoint metrics, secondary endpoints and any combination thereof.

According to some embodiments, the statistical input parameters are selected from target power (chance of succeeding per number of patients), type I error, allocation logic, statistical test and threshold and any combination thereof.

According to some embodiments, defining the improved space of working points comprises selecting clinical trial design parameters optimizing operating characteristics of the clinical trial design and/or clinical and/or statistical input parameters optimizing the power of the ML model.

According to some embodiments, the method further comprises conducting a large plurality of simulations for the identified optimal clinical trial design parameters.

According to some embodiments, in another aspect a method for identifying optimal clinical trial design parameters is presented herein. The method comprises:
(a) receiving a plurality of clinical trial simulations and their associated simulation outcomes for each of a plurality of working points, wherein each working point is selected from a space of working points defined by optional clinical trial design parameters;
(b) training a machine learning (ML) model on the received number of simulations and their simulated outcomes,
(c) applying the trained ML model on additional, non-simulated working points from the space of working points to obtain their respective predicted simulation outcomes, thereby mapping the space of working points;
(d) defining an improved space of working points based on the mapping;
(e) updating the trained ML model, based on predicted simulation outcomes computed for a plurality of working points within the improved space of working points,
(f) repeating steps d-e for the improved space of working points until obtaining an optimal space of working points;
(g) identifying/outputting an optimal set of trial design parameters, based on the predicted simulation outcomes of a plurality of working points from the optimal space of working points.

According to some embodiments, the choice of ML model may depend on the problem. According to some embodiments, the algorithms utilized can vary throughout the process. For example, at early stages a simple model e.g. logistic or linear models can be utilized to aid in guiding the following simulations into the correct part of the design space. As the process continues more sophisticated models (e.g. random forest), using the results of all prior simulations, are trained. The complex models may estimate the performance for non-monotonous parameters, without degrading the overall performance.

According to some embodiments, the method further comprising outputting, for the optimal set of trial design parameters, one or more of: a probability of getting overall trial success, a probability of finding a best treatment as a function of the number of patients included in the trial, estimated distribution of cost and time of the trial overall, estimated distribution of cost and time until identification of failure, estimated distribution of cost and time until identification of success, distribution of estimated treatment effect, distribution of statistical measures.

According to some embodiments, the number of simulations included in the plurality of simulations is predetermined.

According to some embodiments, the number of simulations included in the plurality of simulations is determined based on a number of simulations required to obtain an accuracy above a predetermined threshold.

According to some embodiments, the plurality of simulations comprises between 50 and 5000 simulations.

According to some embodiments, defining the improved space of working points comprises selecting clinical trial design parameters optimizing operating characteristics of the clinical trial and/or clinical and/or clinical trial design parameters optimizing the accuracy of the ML model(s).

According to some embodiments, the method further comprises conducting a large plurality of simulations for the identified optimal set of clinical trial design parameters.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1 schematically shows a flowchart of a method 100 for identifying optimal clinical trial design parameters, according to some embodiments;

FIG. 2 schematically shows a flowchart of a method 200 for identifying optimal clinical trial design parameters, according to some embodiments; and

FIG. 3 schematically shows table 1 which compares the designs of the method presented herein with an alternative method design and a fixed design.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the vicinity of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

According to some embodiments, methods for identifying optimal clinical trial design parameters, are presented herein.

As used herein, the term “clinical trial” refers to prospective biomedical (or behavioral) research studies on human participants designed to answer specific questions about new treatments. They generate data on dosage, safety and efficacy and typically include four phases. According to some embodiments, the clinical trial may be an exploratory phase II clinical trial.

As used herein, the term “trial simulation” refers to the study of the effects of a drug in virtual patient populations using computational mathematical models.

According to some embodiments, the term “arms”, refers to treatment groups of a clinical trial, and may refer to a clinical trial including a single treatment group, two treatment groups (e.g. first medicament and second medicament, first dose and second dose etc.), three treatment groups, four treatment groups, five treatment groups or more. Each possibility is a separate embodiment.

As used herein, according to some embodiments, the term “clinical trial design parameters” refers to the parameters which define the clinical trial, for example, the number of arms being evaluated, primary outcomes, minimal clinical value required for authorization, expected time to clinical results, historical data, etc.

According to some embodiments, the clinical trial design parameters may be statistical parameters and/or clinical parameters that influence the simulation outcome and/or the operating characteristics of the clinical trial.

As used herein, the term “operating characteristics” refers to information on a clinical trial design's expected behavior under specific conditions. A few of the most common measures are the expected probability of success (i.e. power), the expected effect size of the treatment at the end of the trial, the expected duration of the trial, and the required sample size. As used herein, the term “expected” refers to the fact that these operating characteristics are not deterministic and can vary from trial to trial, even when the “truth” is constant.

According to some embodiments the clinical trial design parameters may be classified into three types of parameters:

According to some embodiments the clinical trial design parameters may be internal parameters. As used herein, the term “internal parameters” refers to parameters which have at least one freedom degree, which influence the simulation outcome but are not directly of interest to the client in and of themselves. A non-limiting example of internal parameters is the method used for statistical significance testing as long as it is accepted by the regulatory body.

According to some embodiments the clinical trial design parameters may be reality parameters. As used herein, the term “reality parameters” refers to parameters which cannot be directly controlled, however, affect directly the operating characteristics of the clinical trial. According to some embodiments, the client is required to assume a value of the parameters, but does not directly control the value that will manifest in practice.

A non-limiting example of internal parameters is the efficacy of the medicament tested. According to some embodiments, the reality parameters may be parameters provided by the client. As a non-limiting example, a reality parameter provided by the client may be the recruitment rate of patients.

According to some embodiments the clinical trial design parameters may be external parameters. As used herein, the term “external parameters” refers to parameters which have at least one degree of freedom, and which affect the operating characteristics of the clinical trial. A non-limiting example of an external parameter is the number of interim analyses included in the clinical trial in that each interim analysis may affect the cost of the trial and/or the duration of the trial and therefore the number of interim analysis is limited by the rigidity of the operating characteristics of the clinical trial.

According to some embodiments, the term “optimal parameters” and “optimal design parameters” may be used interchangeably and refer to a set/combination of parameters that significantly (e.g. by at least 0.5% or at least 1%) improve one or more of the operating characteristics of the clinical trial.

As used herein, according to some embodiments, the term “working point” refers to a point where all the clinical trial design parameters are defined. For example, a first working point may be defined as a trial comparing 3 treatment arms of different doses and a control arm, using response adaptive randomization (RAR) with 2 interims, after 40% of patients are recruited and after 70%, using Thompson sampling for re-allocation based on the probability of each arm being best, without futility or efficacy stopping, with control allocation matched to the leading arm, and with acceptance based on a proportion test with a threshold of 0.01 for each arm. The endpoint is binary (response) and the assumed effect size is from a binomial distribution with a probability of 0.1 for the placebo, 0.15 for arm 1, 0.2 for arm 2 and 0.3 for arm 3. The expected recruitment rate is 1 patient per site per month with an s profile (slow recruitment initially, fast in the mid stage and slow again towards the end of the trial), and with a dropout of 15%. The required power is 0.8 and the required family wise type I error rate is 0.025.

As used herein, according to some embodiments, the term “a space of working points” refers to the space generated by a large number of working points each defined by a different set/combination of clinical trial design parameters.

As used herein, according to some embodiments, the term “interim analysis” refers to an analysis of data from an ongoing trial before data collection has been completed. The number of interim as well as their timing is determined prior to commencing the trial. According to some embodiments, interim analysis results may cause modifications in the conduct of the trial. Depending on the results, an interim analysis may lead to changes, such as stopping one treatment arm or changing the number of participants in a group, or stopping the trial altogether.

As used herein, the term “minimal clinical value” refers to the minimal effect of the medicament that has clinical value. For example, in pancreatic cancer any drug prolonging life expectancy by a month may be considered to have clinical value due to the short overall life expectancy of pancreatic cancer patients.

As used herein, the term “assumed clinical value” refers to the effect that the client expects to observe in the trial, e.g., due to previous clinical data or pre-clinical data. For example, in pancreatic cancer while any drug prolonging life expectancy by a month may be considered to have clinical value, the client may assume the effect will be substantially larger based on previous clinical or pre-clinical data possessed and design the trial powered to detect this effect size.

According to some embodiments, the herein disclosed method may include the following steps:

Step 1: Randomly sample a set of design configurations from a relevant range of parameters. These are sampled based on optimal experimental design concepts to remain within reasonable bounds, while still optimizing the expected information gain (i.e. more samples at the edge of the design space).

Step 2: Running a small number of simulations for each of the configurations (can be as few as a single simulation). These simulations output both the binary outcome-success or failure, but also a more detailed output, i.e the test statistic, the effect size to support better training of the ML model.

Step 3: Train a flexible machine learning model on the results of the simulations to estimate the relationship between the configuration and the operating characteristics. The choice of the specific model may depend on the problem. Moreover, different algorithms can be applied and can vary throughout the process. For example, at early stages a simple model (such as logistic or linear models) is utilized to aid in guiding the following simulations into the correct part of the design space. As the process continues, more sophisticated models, using the results of all prior simulations, can be trained. Advantageously, these models can estimate the performance for non-monotonous parameters, without degrading the overall performance.

Step 4: Estimating the uncertainty of the model. According to some embodiments, the estimation of the uncertainty can be based on analytical results for some of the models, while a more complex Bayesian approach can be applied for more sophisticated models (utilizing the model uncertainties).

According to some embodiments, steps 1-4 are run iteratively. Each time the newly sampled points are nearer the optimal design and the ML model fits better to the optimal set of parameters and the expected uncertainty decreases, such that it converges to the required design (optimal design).

Step 5: Confirming the statistical performance of the identified configuration by running a large number of simulations at the chosen point.

The process can terminate after a given number of simulations, a given number of iterations or after converging to the required accuracy.

According to some embodiments, the process can run while a wide range of assumptions are still examined, and these can be included into the ML model parameters. As a result, the optimal point may be based on a range of assumptions rather than a single point.

Advantageously, when comparing the number of iterations required for the herein disclosed model to that of brute force search, approximately 25 times fewer simulations are required.

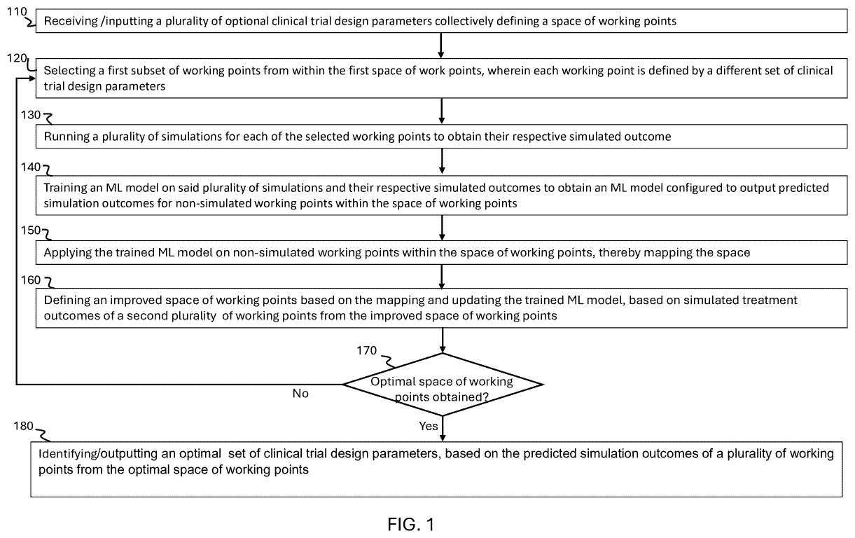

Reference is now made to FIG. 1 which schematically shows a flowchart of a method 100 for identifying optimal clinical trial design parameters, according to some embodiments.

In step 110, a plurality of optional clinical trial design parameters collectively defining a space of working points is received and/or inputted. According to some embodiments, the optional clinical trial design parameters include clinical and statistical input parameters.

In step 120, according to some embodiments, a first subset of working points from within the first space of working points is selected, wherein each working point is defined by a different set of clinical trial design parameters. According to some embodiments, the selection of working points in step 120 is given, random and/or computed. According to some embodiments, the first subset of working points includes between 80-5,000, or between 80-1,000 or between 80-500 working points Each possibility is a separate embodiment.

According to some embodiments, in step 130, a plurality of simulations for each of the selected working points is run to obtain their respective simulated outcome. According to some embodiments, the number of simulations included in the plurality of simulations is predetermined. According to some embodiments, the number of simulations included in the plurality of simulations is determined based on a number of simulations required to obtain an accuracy above a predetermined threshold. According to some embodiments, the plurality of simulations comprises between 50 and 5000 simulations, between 100 and 1000 simulations or between 100 and 500 simulations. Each possibility is a separate embodiment.

According to some embodiments, in step 140, a machine learning (ML) model is trained on said plurality of simulations and their respective simulated outcomes, to obtain an ML model configured to output predicted simulation outcomes for additional (simulated or non-simulated) working points within the space of working points. According to some embodiments, the ML model is a logistic regression, according to some embodiments the ML model is a random forest classifier, according to some embodiments the ML model is a gaussian process regression, according to some embodiments the ML model is a boosted regression tree, according to some embodiments the ML model is a regularized GLM, according to some embodiments the ML model is a neural network. According to some embodiments, various models are applied sequentially. As a non-limiting example a logistic model may initially be applied followed by utilization of more complex models, such as but not limited to random forest models.

In step 150, according to some embodiments, the trained ML model is applied on additional non-simulated working points (and optionally also on the simulated working points), to obtain their respective predicted simulation outcomes, thereby mapping the space of working points.

In step 160, according to some embodiments, an improved space of working points is defined based on the mapping, e.g. according to the contributive predictive power of regions of the space. For example, the predictive power of the ML model may not be expected to improve by including a 4th and 5th interim analyses, and the second space of working point may therefore exclude working points that include 4th and 5th interim analyses.

Then, a second plurality of simulations is, according to some embodiments, then conducted on working points from the second space of working points, and the trained ML model is updated, based on the simulated treatment outcomes (operating characteristics). As a non-limiting example a logistic model may initially be applied followed by utilization of more complex models, such as but not limited to random forest models.

According to some embodiments, step 170, further includes checking whether an optimal space of working points is obtained. According to some embodiments, the optimality is predefined and combines different relevant parameters. In case an optimal space of working points is not obtained, steps 120-170 may be repeated until obtaining an optimal space of working points.

When an optimal space of working point is obtained, according to some embodiments, at step 180, an optimal set of clinical trial design parameters is identified/outputted, based on the predicted simulation outcomes conducted on a plurality of working points from the optimal space of working points. According to some embodiments the optimal space of working points may be encompassed by the first space of working points. According to some embodiments, the optimal space of working points may be only partially encompassed by the first space of working points.

According to some embodiments, defining the optimal set of clinical trial design parameters includes optimizing sample size, cost of the clinical trial, duration of the clinical trial, estimated treatment efficacy of the trial, probability of success of the trial or any other operating characteristic or combination thereof. Each possibility is a separate embodiment.

According to some embodiments, method 100 further includes a step of outputting, for the optimal set of trial design parameters, one or more of: a probability of overall trial success, a probability of finding a best treatment as a function of the number of patients included in the trial, estimated distribution of cost and time of the trial overall, estimated distribution of cost and time until identification of failure, estimated distribution of cost and time until identification of success, distribution of estimated treatment effect, distribution of statistical measures. Each possibility is a separate embodiment.

According to some embodiments, at least a portion of the clinical and/or statistical input parameters include value ranges. According to some embodiments, the value ranges may be predetermined. According to some embodiments, the value ranges may be determined during the simulation and may change due to simulation progress. Additionally or alternatively, method 100 further comprises determining/computing suitable ranges for the portion of clinical and/or statistical input parameters.

According to some embodiments, the clinical parameters are selected from: primary endpoint which is the main result at the end of the trial to see if a given treatment worked, delay (reality parameter), number of arms included (external parameter), futility threshold efficacy (i.e. how bad does the treatment output need to be to stop the trial (internal parameter)), efficacy threshold (how good does the treatment output need to be before deciding success (internal parameter)) assumed clinical efficacy, recruitment rate, primary endpoint metrics (the metrics for measuring success (external parameters)), secondary endpoints (which may provide supportive information about a treatment's effect on the primary endpoint or demonstrate additional effects on the disease or condition) and any combination thereof.

According to some embodiments, the statistical input parameters are selected from: target power (chance of succeeding per number of patients), allocation logic, statistical test and any combination thereof.

According to some embodiments, defining the improved space of working points comprises selecting clinical trial design parameters optimizing operating characteristics of the clinical trial design and/or clinical and/or statistical input parameters optimizing the power of the ML model.

According to some embodiments, the method further comprising conducting a large plurality of simulations for the identified optimal clinical trial design parameters. According to some embodiments, the large plurality of simulations may include at least 50,000 simulations or at least 100,000 simulations per working points from the optimal space of working points.

According to some embodiments, the method further comprises running 100000 simulations after identifying/outputting the optimal set of clinical trial design parameters, as may be required by regulations, or any other number of simulations that may be required by regulations.

According to some embodiments, the ML model capabilities may be expanded to integrate real-time data from ongoing trials, thereby further optimize trial designs adaptively, making them more responsive to interim results and external factors.

According to some embodiments, a method for identifying optimal clinical trial design parameters is presented.

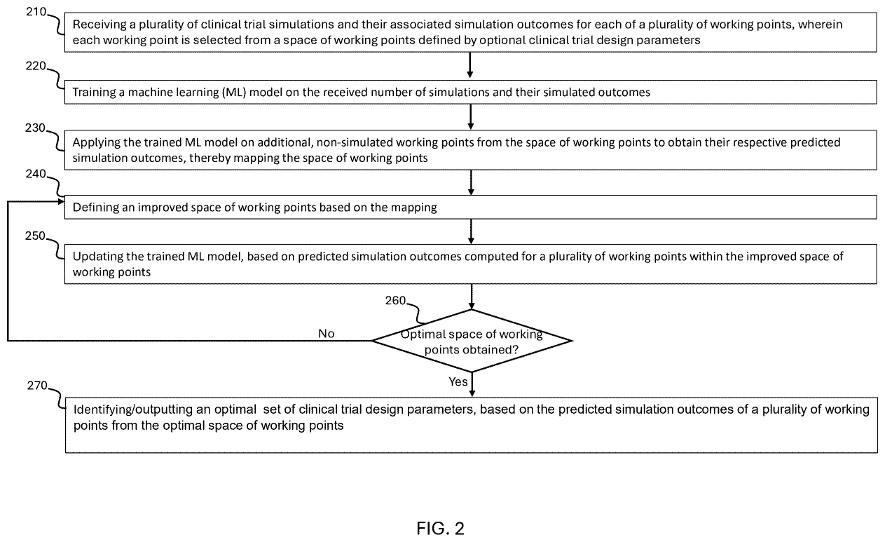

Reference is now made to FIG. 2 which schematically shows a flowchart of a method 200 for identifying optimal clinical trial design parameters, according to some embodiments.

In step 210, according to some embodiments, a plurality of clinical trial simulations and their associated simulation outcomes for each of a plurality of working points are received, wherein each working point is selected from a space of working points defined by optional clinical trial design parameters.

In step 220 according to some embodiments, a machine learning (ML) model is trained on the received number of simulations and their simulated outcomes, to obtain an ML model configured to output predicted simulation outcomes for additional (simulated or non-simulated) working points within the space of working points. According to some embodiments, the ML model is a logistic regression, according to some embodiments the ML model is a random forest classifier, according to some embodiments the ML model is a gaussian process regression, according to some embodiments the ML model is a boosted regression tree, according to some embodiments the ML model is a regularized GLM, according to some embodiments the ML model is a neural network. According to some embodiments, various models are applied sequentially. As a non-limiting example, a logistic model may initially be applied followed by utilization of more complex models, such as but not limited to random forest models.

In step 230, according to some embodiments, the trained ML model is applied on additional, non-simulated working points from the space of working points to obtain their respective predicted simulation outcomes, thereby mapping the space of working points.

In step 240, according to some embodiments, an improved space of working points is defined based on the mapping, as essentially described herein above.

In step 250, according to some embodiments, the trained ML model is updated based on simulation outcomes computed for a plurality of working points within the improved space of working points. According to some embodiments, the updating further comprises exchanging one type of model with another (e.g. logistic model with random forest model)

According to some embodiments, in step 260, it is checked whether an optimal space of working points is obtained. In case an optimal space of working points is not obtained, steps 240-250 may be repeated for the improved space of working points until obtaining an optimal space of working points.

When an optimal space of working points is obtained, an optimal set of trial design parameters is identified/outputted (at step 270), based on the predicted simulation outcomes conducted on a plurality of working points from the optimal space of working points. According to some embodiments the optimal space of working points may be encompassed by the first space of working points. According to some embodiments, the optimal space of working points may be only partially encompassed by the first space of working points.

According to some embodiments, defining the optimal set of clinical trial design parameters includes optimizing sample size, cost of the clinical trial, duration of the clinical trial, estimated treatment efficacy of the trial, probability of success of the trial or any other operating characteristic or combination thereof. Each possibility is a separate embodiment.

According to some embodiments method 200 further comprising outputting, for the optimal set of trial design parameters, one or more of: a probability of getting overall trial success, a probability of finding a best treatment as a function of the number of patients included in the trial, estimated distribution of cost and time of the trial overall, estimated distribution of cost and time until identification of failure, estimated distribution of cost and time until identification of success, distribution of estimated treatment effect, distribution of statistical measures. Each possibility is a separate embodiment.

According to some embodiments, the number of simulations included in the plurality of simulations is predetermined. According to some embodiments, the number of simulations included in the plurality of simulations is determined based on a number of simulations required to obtain an accuracy above a predetermined threshold. According to some embodiments, the plurality of simulations comprises between 50 and 5000 simulations, between 100 and 1000 simulations or between 100 and 500 simulations. Each possibility is a separate embodiment.

According to some embodiments, defining the improved space of working points comprises selecting clinical trial design parameters optimizing operating characteristics of the clinical trial and/or clinical and/or clinical trial design parameters optimizing the power of the ML model.

According to some embodiments, the method further includes conducting a large plurality of simulations for the identified optimal set of clinical trial design parameters. According to some embodiments, the large plurality of simulations may include at least 50,000 simulations or at least 100,000 simulations per working points from the optimal space of working points.

According to some embodiments, the method further comprises running 100000 simulations after identifying/outputting the optimal set of clinical trial design parameters, as may be required by regulations, or any other number of simulations that may be required by regulations.

According to some embodiments, the ML model capabilities may be expanded to integrate real-time data from ongoing trials, thereby further optimize trial designs adaptively, making them more responsive to interim results and external factors.

According to some embodiments, a method for identifying optimal clinical trial design parameters is presented.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1

An example of a use case is now presented herein, of a sponsor (also referred to herein as “client”) in the process of developing a novel peptide for a rheumatic disease. The sponsor is interested in examining the potential benefits of an adaptive design.

### Clinical Trial Design Parameters:

The trial was a phase 2 trial, with multiple doses and treatment regiments. The sponsor was also interested in exploring a potential combination with an existing drug.

A set of 4 treatment arms, as well as a control arm (that would allow the sponsor to obtain the information required for phase 3) were examined.

The Estimated effect size in the control arm was expected to be low (5-10%) based on previous data.

The assumed effect size for the treatment was 30% per arm, and the sample size was calculated to allow for 80% power and a type I error of 2.5%.

The estimated recruitment rate was high (30 patients per month).

The expected time to measure the endpoint for supporting adaptation was 1 month.

Based on the above, the most relevant degrees of freedom in the trial were:
1. Number of interim analysis (1-5)
2. Timing of the interim analysis (any time after 2 months until 5 months)
3. Maximal sample size (from 100 to 300)
4. Test statistic threshold for efficacy at the end of the trial (up to 0.025).
5. Type of adaptive design (RAR, GSD, SSR, combinations thereof)
6. Aggressiveness (i.e. focus on the “best” arm or on “any promising” arm.)

For each of the input parameters mentioned above, there are many additional potential configurations (for example each interim can be at any time within the range mentioned, for simplicity it is assumed, equally spaced interims from the first interim to the end of the trial).

In addition, the sponsor was interested in assessing the performance of the design under higher efficacy assumptions (effect size of 0.45), and lower efficacy assumptions (0.2). The sponsor also presented a number of possible scenarios—a single effective treatment arm, a scenario with two effective arms, and a scenario where all the arms are at least partially effective.

In addition, the selected scenario was further assessed under a slower recruitment rate, which better aligned with historical precedent.

The sponsor also wanted to consider a design with a strict type I family-wise error control, as it would better serve as supporting evidence in a regulatory setting.

Accordingly, even at this high level of initial designing of the trial, the number of potential configurations is very large: assuming only 5 options for each of the input parameters that are examined the total number of combinations is:

5×5×5×5×5×5=15,625

All these need to be examined under 3×3×2×3 i.e. 54 assumptions. Thus, in the absence of the herein disclosed method and system almost a million optional combinations would need to undergo the 100K simulations required by regulation bodies.

In addition, eventually, the sponsor decided to examine an additional treatment arm, which in the absence of the herein disclosed method and system would require that the whole process be rerun.

### Results

The method presented herein was applied on the initial trial design, described above. Given the large number of degrees of freedom, the initial phase included running simulations for 2-3 configurations of each of the input parameters-thus decreasing the number of combinations by 3 orders of magnitude. Moreover, for each of these configurations only 10 simulations were run, thus decreasing the total number of simulations by 4 more orders of magnitude.

This initial step allowed getting a very rough estimate of the range of operating characteristics, using a simple logistic model. This model was only aimed at assessing the directional impact of each parameter to support more relevant sampling at the next iteration.

Then about 150 additional working points were simulated, with 50 simulations each, focusing on the most promising areas of the space of working points (2-3 interims, max sample size of 230, test statistic in the range of 0.015 to 0.02, RAR design).

Advantageously, after completing 6 iterations, and gradually introducing more sophisticated ML models (e.g. starting with logistic and then random forest), an optimal set of operating parameters was outputted namely: an 33% saving, reduced sample size (175 as opposed to 240) utilizing response adaptive randomization, with 2 interims, at 35% and 68% of the trial, with a maximal sample size of 175 patients, using a less aggressive adaptation at the first interim, and more aggressive adaptation at the second interim.

Moreover, it was able to assess the performance under different scenarios and assumptions.

Finally, 100,000 simulations were run at the proposed design configuration, which simulation validated the result. Moreover, a set of simulations with 10,000 runs at 3 additional assumptions was also conducted and the robustness of the design was validated.

The proposed design almost quadrupled the expected savings the sponsor was suggested by an alternative method.

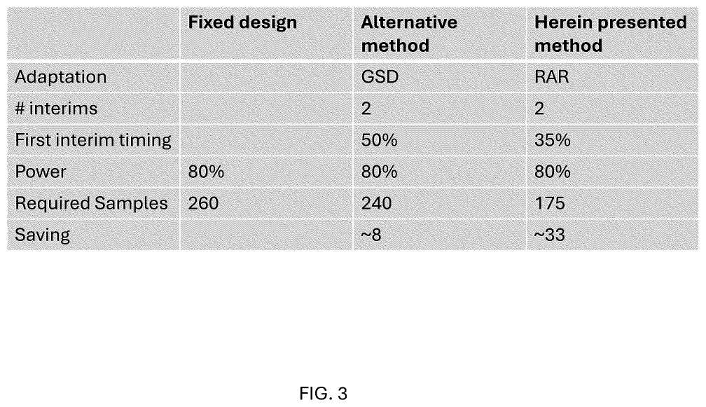

FIG. 3 schematically shows a table, which compares the designs of the method presented herein with an alternative method design and a fixed design (see Hartung J. Biom J. 2006 August; 48 (4): 521-36.).

As can be seen from the table, while in the fixed design only the power and the required samples are defined and no adaptation is/can be applied, the alternative method applies a group sequential design (GSD) adaptation and requires 240 samples, 2 interims and a first interim at 50% of the trial, which leads to a saving of 8%. The method presented herein, applies a response adaptive randomization (RAR) and defines 2 interims with the first interim at 35% of the trial, only 175 required samples, leads to a saving of 33%, thus clearly indicating its superiority.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods, according to some embodiments, may be described in a specific sequence, the methods of the disclosure may include some or all of the described stages carried out in a different order. In particular, it is to be understood that the order of stages and sub-stages of any of the described methods may be reordered unless the context clearly dictates otherwise, for example, when a later stage requires as input an output of a former stage or when a later stage requires a product of a former stage. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications, and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Section headings are used herein to ease understanding of the specification and should not be construed as necessarily limiting.

1. A method for identifying optimal clinical trial design parameters, the method comprising:
a. inputting a small plurality of optional clinical trial design parameters collectively defining a space of working points;
b. selecting a first subset of working points from within the first space of working points, wherein the first subset of working points comprises 80-5000 of working points out of the totality of working points in the space, wherein each working point is defined by a different set of clinical trial design parameters;
c. running a plurality of simulations for each of the selected working points to obtain their respective simulated outcome, wherein the plurality of simulations comprises between 50 and 5000 simulations;
d. training a machine learning (ML) model on said plurality of simulations and their respective simulated outcomes to obtain a trained ML model configured to output predicted simulation outcomes for non-simulated working points from within the space of working points, wherein the ML model is selected from a logistic regression model, a random forest classifier, a gaussian process regression, a boosted regression tree, a regularized GLM, and/or a neural network model while running simulations only on the subset of working points;
e. applying the trained ML model on non-simulated working points from within the space of working points, thereby mapping the space;
f. defining an improved space of working points based on the mapping;
g. selecting a second subset of working points from within the improved space of working points;
h. running a second plurality of simulations on the second subset of working points;
i. updating the trained ML model, based on simulated treatment outcomes of the second plurality simulations to obtain an updated trained ML model;
j. repeating steps d-i until obtaining an optimal working point comprising a defined set of clinical trial design parameters achieving a predetermined required predictive accuracy with at least 25 times fewer simulations as compared to brute force methods; and
k. outputting a clinical trial design comprising the defined set of clinical trial design parameters.

2. The method of claim 1, further comprising running up to 100000 simulations on the optimal set of clinical trial design parameters.

3. The method of claim 1, wherein outputting an optimal set of clinical trial design parameters comprises optimizing sample size, cost of the clinical trial, duration of the clinical trial, estimated treatment efficacy of the trial, probability of success of the trial or any combination thereof.

4. The method of claim 1, further comprising outputting, for the optimal set of trial design parameters, one or more of: a probability of overall trial success, a probability of finding a best treatment as a function of the number of patients included in the trial, estimated distribution of cost and time of the trial overall, estimated distribution of cost and time until identification of failure, estimated distribution of cost and time until identification of success, distribution of estimated treatment effect, distribution of statistical measures.

5. The method of claim 1, wherein at least a portion of the clinical design input parameters comprise value ranges.

6. The method of claim 5, wherein the value ranges are predetermined.

7. The method of claim 5, wherein the method further comprises determining/computing suitable ranges for the portion of clinical and/or statistical input parameters.

8. The method of claim 1, wherein the selection of working points of step (b) is given and/or computed.

9. The method of claim 1, wherein the number of simulations included in the plurality of simulations is predetermined.

10. The method of claim 1, wherein the number of simulations included in the plurality of simulations is determined based on a number of simulations required to obtain an accuracy above a predetermined threshold.

11. (canceled)

12. The method of claim 1, wherein optional clinical trial design parameters comprise clinical and statistical input parameters.

13. The method of claim 12, wherein the clinical parameters are selected from primary endpoint, delay, number of arms, futility threshold efficacy, efficacy threshold, assumed clinical efficacy, recruitment rate, primary endpoint metrics, secondary endpoints and any combination thereof.

14. The method of claim 12, wherein the statistical input parameters are selected from target power (chance of succeeding per number of patients), allocation logic, statistical test and any combination thereof.

15. The method of claim 1, wherein defining the improved space of working points comprises selecting clinical trial design parameters optimizing operating characteristics and/or clinical and/or statistical input parameters optimizing a power of the ML model.

16. The method of claim 1, further comprising conducting a large plurality of simulations for the identified optimal clinical trial design parameters.

17.-23. (canceled)

24. The method of claim 1, wherein the ML model comprises a random forest model.

25. The method of claim 1, wherein the ML model comprises a simple ML model in step e and a complex model in at least some of the repeating of step j.

26. The method of claim 25, wherein the simple model comprises a logistic model or a linear model and the complex model is random forest classifier, a gaussian process regression, a boosted regression tree, a regularized GLM, and/or a neural network model.

27. The method of claim 25, wherein the simple model comprises a logistic model and the complex model is random forest classifier.

28. The method of claim 1, wherein the first subset of working points comprises 80-1000 of working points.
